(19) United States
(12) Patent Application Publication
(10) Pub. No.: US 2009/0312617 A1
(43) Pub. Date: Dec. 17, 2009

(54) NEEDLE INJECTION CATHETER

(76) Inventors: Jerett Creed, Hermosa Beach, CA (US); Emerson Perin, Houston, TX (US); Knut Sauerteig, Purgen (DE); Juan Granada, Orangeburg, NY (US)

Correspondence Address: BELASCO, JACOBS & TOWNSLEY LLP;HOWARD HUGHES CENTER, 6701 CENTER DRIVE WEST, 14th Floor, LOS ANGELES, CA, 90045, US

(21) Appl. No.: 12/249,826

(22) Filed: Oct. 10, 2008

Related U.S. Application Data
(63) Continuation-in-part of application No. 12/138,201, filed on Jun. 12, 2008.

(51) Int. Cl. A61B 5/042; A61B 5/055; A61B 18/18; A61B 8/14; A61M 1/00

(52) U.S. Cl. 600/345; 600/509; 600/374; 600/424; 606/41; 600/464; 604/27

(57) ABSTRACT

The needle injection catheter includes a delivery tube and at least one hypotube that fits slidably within the delivery tube. The hypotube has at least three hollow needle portions extending outwardly at its distal end. The needle portions curve outwardly and have ends shaped to penetrate tissue. At least one reference electrode is located on the delivery tube, spaced from the second end. At least one proximal electrode is located adjacent and spaced from the end of the needle portion. The proximal electrode is electrically connected to a first notification device. A microcircuit is electrically connected to the proximal electrode, the reference electrode and to a power supply. A distal electrode is located adjacent the end of the needle portion and electrically connected to the microcircuit. A tip electrode is located adjacent the second end of the delivery tube and electrically connected to a second notification device and the microcircuit.

### RELATED APPLICATION

This application is a Continuation-in-Part of U.S. application Ser. No. 12/138,201, filed Jun. 12, 2008 and incorporates by reference the disclosure thereof.

### FIELD OF INVENTION

This invention relates to the field of stem cell therapies, and more specifically to apparatus and methods for safely injecting cells and therapeutic materials into heart chamber or other organ walls.

### BACKGROUND OF THE INVENTION

There has been a significant increase in the amount of research and funding in the area of stem cell based therapies for repairing and treating various diseases. More specifically, the number of clinical trials using cell based approaches to treat cardiac related diseases has tripled in the last few years. While much focus has been placed on the particular cell types and mechanisms of action within various tissue types and locations within the heart, very little has been developed with respect to the delivery of cells. Most of the work coming out of Europe has concentrated on the intra coronary route of administration. This has proved to have mixed effects in the acute timeframe (less than 10 days) but poor results in the chronic setting.

Recently, efforts have been placed on the intramyocardial delivery of cells directly into the heart muscle. This is accomplished by accessing the inside chamber of the left ventricle via a retrograde crossing of the aortic valve. Once inside the chamber, the physician will attempt to directly inject a needle-based catheter into the tissue of the endocardial surface and deliver a particular therapy (cell based or otherwise). These needle based catheters are tracked in the vasculature via standard x-ray fluoroscopy which provides only 2D visualization. This visualization is not optimal inside a 3D space particularly given the desire to deliver cells specifically to certain areas of interest.

Manipulating the catheter from outside the body with only a 2 dimensional understanding of the catheter tip movement is not adequate in the long run of cell based delivery locally to the inside chambers of the heart or elsewhere in the body. In addition, it creates safety issues due to applying excessive force on the tip of the catheter and making gross movements of the catheter without knowing the area within the ventricle where the catheter tip is located. This safety issue has become the number one issue for cell injection procedures. What is required is a catheter that is unable to perforate the ventricle or organ wall regardless of the force translated to the tip with the possibility of real time imaging of that catheter utilizing standard x-ray equipment.

What is proposed is a needle based injection catheter that has enhanced safety features such that it is extremely difficult to perforate the myocardium and has diagnostic features adding to the safety profile. In addition, a catheter that with minimal modifications can also be imaged and tracked via a standard single c-arm fluoroscopy system is also described briefly. Various medical devices have been developed to address injecting cells, drugs and other therapeutic means into organs of the body.

U.S. Pat. No. 7,087,040, issued to McGuckin, Jr. et al., discloses a surgical apparatus for delivering fluid to treat a lesion comprising a housing, an elongated member extending from the housing, and a plurality of tines positioned in the housing. Each of the tines has a lumen and at least one opening communicating with the lumen for delivering fluid to the lesion. An actuator is operatively associated with the tines and actuable to a first position to move the plurality of tines from a retracted position substantially within the elongated member to a first deployed position extending from the elongated member and actuable to a second position to move the plurality of tines from the first position to a second deployed position.

U.S. Patent Application No. 2006/0004325, published for Hamatake et al. is directed to multi-lumen catheters with improved tip configurations, including a triple-lumen catheter which may be useful for apheresis. In one variation, the catheter has three lumens with distal openings angularly spaced apart and staggered axially with respect to one another. In another variation, the catheter has two lumens exiting distally and one centrally positioned lumen exiting proximally. A third variation is a catheter with a single distal opening and two proximal openings. The staggered lumen openings along the axial length of the catheter may decrease recirculation while maximizing flow rates.

U.S. Patent Application No. 2005/0228452, published for Mourias et al. illustrates an apparatus for treating tissue that includes a flexible catheter including a proximal end, a distal end for introduction into a chamber of a heart, a transparent balloon carried by the distal end, an optical imaging assembly carried by the distal end for imaging tissue structures beyond the distal end through the balloon, and a needle deployable from the tubular member for penetrating the tissue structure to treat tissue. The apparatus may include a source of stems cells or other therapeutic and/or diagnostic agent coupled to the needle, a guide catheter advanceable over the needle for accessing a region beyond the tissue structure penetrated by the needle, and/or an energy probe deployable from the catheter for delivering electrical energy to tissue in the region beyond the tissue structure. The apparatus may be used to deliver stem cells into infracted tissue or for ablating heart tissue, e.g., from a trans-septal approach.

U.S. Pat. No. 6,302,870, issued to Jacobsen et al. disclose an apparatus for injecting fluids into the walls of blood vessels, body cavities, and the like, includes a plurality of laterally flexible needles disposed in a catheter for exit either out the distal end of the catheter or the catheter or through corresponding side openings in the catheter. In the latter case, the terminal ends of the needles would be curved laterally, with each terminal end being positioned in a respective side opening so that when the needles were moved forwardly in the catheter, the terminal ends of the needles would move laterally out the respective openings to pierce a vessel or cavity wall adjacent to which the catheter was positioned. Hilts positioned near the terminal ends of the needles serve to control the depth of penetration of the needles.

U.S. Patent Application No. 2006/0278248, published for Viswanathan and U.S. Patent Application No. 2007/0179492, published for Pappone are directed to a method of applying an electrode on the end of a flexible medical device to the surface of a body structure, the method including navigating the distal end of the device to the surface by orienting the distal end and advancing the device until the tip of the device contacts the surface and the portion of the device proximal to the end prolapses. Alternatively the pressure can be monitored with a pressure sensor, and used as an input in a feed back control to maintain contact pressure within a pre-determined range.

It is an objective of the present invention to provide a method and apparatus for injecting cells, drugs or other therapeutic agents into heart or other organ walls while minimizing the danger of penetrating those walls with the injection device. It is a further objective to provide a feedback system for the apparatus that will allow a physician to determine the point at which the distal end of the injection apparatus comes in contact with the organ wall. It is a still further objective of the invention to provide more detailed feedback to inform the physician of the point at which the injection needles of the apparatus contact the organ walls. It is yet a further objective to provide an apparatus that can be easily guided to the desired location within the body by means of 2 dimensional X-ray or related scanning technology. In is another objective of the invention that the location of the catheter is able to be tracked both laterally and radially within the body. Finally, it is an objective of the present invention to provide such apparatus that is durable, inexpensive and compatible with standard sterilization procedures.

While some of the objectives of the present invention are disclosed in the prior art, none of the inventions found include all of the requirements identified.

### SUMMARY OF THE INVENTION

The present invention addresses all of the deficiencies of prior art needle injection catheter inventions and satisfies all of the objectives described above.

(1) A needle injection catheter providing the desired features may be constructed from the following components. A delivery tube is provided. The delivery tube has a first end, a second end and a handle portion adjacent the first end. At least one hypotube is provided. The hypotube is sized and shaped to fit slidably within the delivery tube. The hypotube has a proximal end, a distal end and at least three hollow needle portions connected to and extending outwardly at the distal end. The needle portions are formed of resilient material, curving outwardly from a central axis of the delivery tube and have ends shaped to penetrate tissue when the hypotube is urged toward the second end of the delivery tube. A first sensor is provided. The first sensor is located adjacent the second end of the delivery tube and is electrically connected to a first notification device located adjacent the first end. At least one second sensor is provided. The second sensor is located adjacent the end of the needle portion and electrically connected to a second notification device located adjacent the first end of the delivery tube. A microcircuit is electrically connected to the first and second sensors and to a power supply.

(2) In a variant of the invention, the microcircuit and power supply are contained within the handle portion.

(3) In another variant, the first and second sensors detect electrical voltages associated with contact with bodily fluids, tissues and organ walls.

(4) In still another variant, the detected voltages range from 1-30 millivolts.

(5) In yet another variant, the first and second sensors detect temperatures associated with contact with bodily fluids, tissues and organ walls.

(6) In a further variant, the first and second sensors detect chemical compositions associated with contact with bodily fluids, tissues and organ walls.

(7) In still a further variant, the first and second sensors detect a level of PH associated with contact with bodily fluids, tissues and organ walls.

(8) In another variant of the invention, the first and second sensors provide optical imaging to discriminate between bodily fluids, tissues and organ walls.

(9) In still another variant, the first and second sensors detect changes in impedance between bodily fluids, tissues and organ walls.

(10) In yet another variant, the first and second notification devices are either light emitting devices or sound emitting devices.

(11) In a further variant, the second notification device differs from the first notification device to indicate a hazard condition should the first sensor contact tissue.

(12) In still a further variant, the needle portions secure the second end of the delivery tube at a predetermined distance from the tissue penetrated by the needle portions.

(13) In yet a further variant, the second end of the delivery tube is secured at a predetermined distance from the tissue during movement due to cardiac cycles.

(14) In still a further variant, extension of the needle portions from the second end of the delivery tube is limited to at least one predetermined distance by an extension control disposed adjacent the handle portion.

(15) In another variant of the invention, the extension control provides for a predetermined minimum extension of the needle portions beyond the second end of the delivery tube.

(16) In still another variant, the needle portions extend from 2-7 millimeters beyond the second end of the delivery tube.

(17) In yet another variant, the needle portions are located adjacent the second end of the delivery tube at an angle ranging from 5 to 270 degrees to a central axis of the delivery tube. The range varies as the needle portions are extended outwardly from the second end of the delivery tube.

(18) In a further variant, the angular disposition of the needle portions limits a penetration depth for distal ends of the needle portions.

(19) In still a further variant, a lateral movement of the second end of the delivery tube decreases for a specified applied force with extension of the needle portions.

(20) In yet a further variant, a fixed tip is located adjacent the second end of the delivery tube. The fixed tip has an angled portion to facilitate perpendicular contact with the surface being injected.

(21) In another variant of the invention, the angled portion is bent at a point spaced 10 mm to 120 mm from a distal end of the fixed tip with an angulation of 10° to 90°.

(22) In still another variant, the delivery tube further includes a dielectric coated tip. The tip has an uncoated section located at a distal end for direct contact with either body tissues or other bodily surfaces.

(23) In yet another variant, the dielectric coated tip includes polyxylylene polymers.

(24) In a further variant, the needle portions are dielectric coated along their exterior length and not dielectric coated at their distal ends.

(25) In still a further variant, the needle portions include openings at their distal ends and along their exterior length.

(26) In yet a further variant, the second end of the delivery tube includes a magnetic element.

(27) In another variant of the invention, the delivery tube includes at least one ring electrode.

(28) In still another variant, the ring electrode is either a voltage or impedance reference.

(29) In yet another variant, the reference is electrically connected to the microcircuit.

(30) In a further variant, the microcircuit computes impedance from an alternating current between the reference and the second sensor.

(31) In still a further variant, the microcircuit computes impedance from an alternating current between the reference and the first sensor.

(32) In yet a further variant, an external energy source is provided. The energy source is connected to at least one of the needle portions.

(33) In another variant of the invention, the external energy source is selected from the group consisting of radio frequency (RF) energy and laser energy.

(34) In still another variant, at least one of the needle portions is a thermocouple for measuring temperature at a location of the needle portions.

(35) In yet another variant, the needle portions are formed of material selected from the group consisting of metallic material, shape memory metal, duromers, fiber reinforced materials, polymer and polymer material with a highly refractive index capable of transmitting and receiving an optical signal.

(36) In a further variant, the hypotube is connected to either a fixed luer connection or a flexible luer connection adjacent the handle portion to facilitate introduction of an injectate.

(37) In still a further variant, either a fixed or deflectable tip is located adjacent the second end of the delivery tube.

(38) In yet a further variant, at least one radiopaque marker is located either upon or within the delivery tube.

(39) In another variant of the invention, the radiopaque marker is located adjacent the second end of the delivery tube.

(40) In still another variant, the radiopaque markers are differentiated so that the location of each marker relative to other markers and the second end of the delivery tube is determined.

(41) In yet another variant, the radiopaque markers are located circumferentially either upon or within the delivery tube and are tapered in form to indicate the radial position of the delivery tube when viewed with a radiological scanning system.

(42) In a further variant, at least one radiopaque marker is located adjacent the end of at least one of the needle portions.

(43) In still a further variant, the radiopaque markers are differentiated for each of the needle portions, thereby permitting an operator to identify a location of each needle portion.

(44) In yet a further variant, the needle portions includes a nano-particle enhanced radiopaque coating.

(45) In another variant of the invention, the needle portions include a nano-particle enhanced radiopaque coating mixed with a dielectrtic coating.

(46) In still another variant, any of the first and second sensors are radiopaque markers.

(47) In yet another variant, the tip is a radiopaque marker.

(48) In a further variant, the tip includes a nano-particle enhanced radiopaque coating.

(49) In still a further variant, the tip includes a nano-particle enhanced radiopaque coating mixed with a dielectrtic coating.

(50) In yet a further variant, the tip is rounded or flat for use in ablation.

(51) In another variant of the invention, the second end of the delivery tube includes an ultrasound transducer.

(52) In still another variant, the second end of the delivery tube includes an RF transmitter.

(53) In yet another variant, the second end of the delivery tube includes an electromagnetic coil.

(54) In a further variant, the second end of the delivery tube includes a transponder.

(55) In still a further variant, the delivery tube encloses a single hypotube. The single hypotube is divided into at least three communicating needle portions at the distal end.

(56) In yet a further variant, the delivery tube encloses at least three hypotubes. Each of the hypotubes has a needle portion at the distal end and a separate connection point at the proximal end.

(57) In another variant of the invention, the delivery tube encloses at least three hypotubes. Each of the hypotubes has a needle portion at the distal end and joins into a common connection point at the proximal end.

(58) In still another variant, the hypotube is formed of polymer material with a highly refractive index capable of transmitting and receiving an optical signal.

(59) In yet another variant, the hypotube is connected to a source of laser energy and a microprocessor.

(60) In a further variant of the invention, the needle portions include openings at their distal ends and along their exterior length. A first insulating coating is provided. The first insulating coating extends from the proximal end to a point adjacent a distal end of the needle portion. A first conducting coating is provided. The first conducting coating extends from the proximal end to a point adjacent a distal end of the first insulating coating. A second insulating coating is provided. The second insulating coating extends from the proximal end to a point adjacent a distal end of the first conducting coating. A second conducting coating is provided. The second conducting coating extends from the proximal end to a point adjacent the opening along the exterior length of the needle portion. A third insulating coating is provided. The third insulating coating extends from the proximal end to a point adjacent a distal end of second conducting coating. Each of the insulating and conducting coatings has an aperture sized and shaped to surround the opening along the exterior length of the needle portion. The first and second conducting coatings are connected to the power supply and a pulsing switch. When a current is introduced to the conducting coatings, a magnetic field is established adjacent the openings at the distal ends and along the exterior length of the needle portion.

(61) In still a further variant, a needle injection catheter, includes a delivery tube. The delivery tube has a first end, a second end and a handle portion adjacent the first end. At least one hypotube is provided. The hypotube is sized and shaped to fit slidably within the delivery tube. The hypotube has a proximal end, a distal end and at least three hollow needle portions connected to and extending outwardly at the distal end. The needle portions are formed of resilient material, curving outwardly from a central axis of the delivery tube and have ends shaped to penetrate tissue when the hypotube is urged toward the second end of the delivery tube. At least one reference electrode is provided. The reference electrode is located on the delivery tube, spaced from the second end. At least one proximal electrode is provided. The proximal electrode is located adjacent and spaced from the end of the needle portion. The proximal electrode is electrically connected to a first notification device located adjacent the first end of the delivery tube. A microcircuit is electrically connected to the proximal electrode, the reference electrode and to a power supply.

(62) In yet a further variant, the microcircuit and the power supply are contained within the handle portion.

(63) In another variant of the invention, the microcircuit and the power supply are wirelessly connected to the catheter.

(64) In still another variant, the microcircuit is both analog and digital.

(65) In yet another variant, at least one distal electrode is provided. The distal electrode is located adjacent the end of the needle portion and electrically connected to the microcircuit.

(66) In a further variant, a tip electrode is provided. The tip electrode is located adjacent the second end of the delivery tube and electrically connected to a second notification device located adjacent the first end of the delivery tube and connected to the microcircuit.

(67) In still a further variant, the at least one reference electrode is detected by impedance based mapping systems.

(68) In yet a further variant, the reference electrode is formed of a mixture of platinum and iridium.

(69) In another variant of the invention, impedance is measured between 1 KHz and 75 KHz.

(70) In still another variant, voltage is measured between 1-50 millivolts.

(71) In yet another variant, the delivery tube is insulated.

(72) In a further variant, the needle portions are insulated with polyxylylene polymers.

(73) In still a further variant, the needle portions are coated with 1-40 microns of insulation.

(74) In yet a further variant, wires connected to the needle portions are insulated with polyxylylene polymers.

(75) In another variant of the invention, the proximal electrode is spaced from the end of the needle portion by a first predetermined distance for entry of the needle portion into tissue.

(76) In still another variant, the first notification device is activated only when all three of the needle portions have the proximal electrode in contact with tissue.

(77) In yet another variant, the second notification device is activated only when the tip electrode is embedded is tissue for a first predetermined depth.

(78) In a further variant, the first predetermined depth is 0.5-4 mm.

(79) In still a further variant, the needle injection catheter includes a pulse generator. The pulse generator is electrically connected to a switch. The switch selects between impedance measuring, voltage measuring, and pulse generation.

(80) In yet a further variant, the pulse generator produces a pulse ranging from 1-700 volts for 100 microseconds to 10 milliseconds at up to 200 mA.

(81) In another variant of the invention, the pulse generator creates an electrical field between the proximal electrode and the distal electrode on each of the needle portions.

(82) In still another variant, the pulse generator creates an electrical field between the proximal electrode and the distal electrode on each of the needle portions successively.

(83) In yet another variant, the pulse generator is gated to an electrocardiogram (EKG) device to prevent pulse generation during vulnerable portions of cardiac cycles.

(84) In a further variant, impedance between the proximal electrode and the distal electrode is simultaneously measured for each of the needle portions.

(85) In still a further variant, the needle portions include openings along their exterior length. The openings extend from the proximal electrode toward the end of the needle portion.

(86) In yet a further variant, the needle portions include openings along their exterior length, the openings extending from the proximal electrode to the distal electrode.

(87) In another variant, each of the needle portions has an additional opening at the end of the needle portion.

(88) In a final variant, each of the needle portions has an additional opening at the end of the needle portion.

An appreciation of the other aims and objectives of the present invention and an understanding of it may be achieved by referring to the accompanying drawings and the detailed description of a preferred embodiment.

### DESCRIPTION OF THE DRAWINGS

FIG. 1 is a side elevational view of the preferred embodiment of the invention including a schematic representation of a microcircuit and power supply connected to first and second sensors and notification devices;

FIG. 2 is an enlarged, detailed side elevational view of the first end of the delivery tube of the FIG. 1 embodiment, illustrating the microcircuit and power supply embedded in the handle portion;

FIG. 3 is a front side elevation view of a man, illustrating the relative locations of the heart, aorta and femoral arteries and point of introduction of the needle injection catheter;

FIG. 4 is a partial cross-sectional view of a heart, illustrating the path for introduction of the catheter into the left ventricle of the heart;

FIG. 5 is an enlarged view of the left ventricle illustrating the insertion of the needle portions into the heart wall;

FIG. 6 is an enlarged, detailed side elevational view of a fixed tip of the delivery tube illustrating a predetermined distance that the needle portions will allow the tip to approach body tissue;

FIG. 7 is an enlarged, detailed side elevational view of a fixed tip of the delivery tube illustrating an angled portion, an angled dispersion of the needle portions from each other and a limited predetermined distance that the needle portions may extend from the tip;

FIG. 8 is an enlarged, detailed side elevational view of a fixed tip of the delivery tube illustrating a dielectric coated tip;

FIG. 9 is an enlarged, detailed side elevational view of a distal end of one of the needle portions of the FIG. 1 embodiment illustrating a second sensor;

FIG. 10 is an enlarged, detailed side elevational view of a distal end of one of the needle portions illustrating a dielectric coated length and uncoated tip;

FIG. 11 is an enlarged, detailed side elevational view of a distal end of one of the needle portions illustrating openings along its length;

FIG. 12 is an enlarged, detailed side elevational view of a fixed tip of the delivery tube illustrating a magnetic element;

FIG. 13 is an enlarged, detailed side elevational view of a fixed tip of the delivery tube illustrating a ring electrode;

FIG. 14 is an enlarged, detailed side elevational view of a distal end of one of the needle portions illustrating a thermocouple adjacent the distal end;

FIG. 15 is an enlarged, detailed side elevational view of the handle portion illustrating a fixed luer connection;

FIG. 16 is an enlarged, detailed side elevational view of a deflectable tip of the delivery tube;

FIG. 17 is an enlarged, detailed side elevational view of a fixed tip of the delivery tube illustrating radiopaque markers;

FIG. 18 is an enlarged, detailed side elevational view of the distal ends of three of the needle portions illustrating differentiated radiopaque markers;

FIG. 19 is an enlarged, detailed side elevational view of a distal end of one of the needle portions illustrating a nano-particle enhanced radiopaque coating mixed with a dielectric coating;

FIG. 20 is an enlarged, detailed side elevational view of a fixed tip of the delivery tube illustrating a nano-particle enhanced radiopaque coating;

FIG. 21 is an enlarged, detailed side elevational view of a fixed tip of the delivery tube illustrating a nano-particle enhanced radiopaque coating mixed with a dielectric coating;

FIG. 22 is an enlarged, detailed side elevational view of a fixed tip of the delivery tube illustrating a flattened tip used for ablation;

FIG. 23 is an enlarged, detailed side elevational view of a fixed tip of the delivery tube illustrating an included ultrasound transducer;

FIG. 24 is an enlarged, detailed side elevational view of a fixed tip of the delivery tube illustrating an included RF transmitter;

FIG. 25 is an enlarged, detailed side elevational view of a fixed tip of the delivery tube illustrating an included electromagnetic coil;

FIG. 26 is an enlarged, detailed side elevational view of a fixed tip of the delivery tube illustrating an included transponder;

FIG. 27 is a side elevational view of an alternate embodiment of the catheter having three separate hypotubes each having a separate connection point at the proximal end;

FIG. 28 is a side elevational view of another alternate embodiment of the catheter having a single hypotube divided into three separate needle portions at the distal end;

FIG. 29 is a detailed, partial, side elevational cross-sectional view of a needle portion illustrating openings along the length of the needle and insulating and conducting layers on the needle as well as features for voltage and impedance measurement and pulse generation;

FIG. 30 is a side elevational view of the needle injection catheter illustrating the proximal and reference electrodes;

FIG. 31 is a side elevational view of the handle portion illustrating the enclosed microcircuit and power supply;

FIG. 32 is a side elevational view of the needle injection catheter illustrating the proximal and tip electrodes;

FIG. 33 is a side elevational view of the needle injection catheter illustrating the distal and tip electrodes;

FIG. 34 is an enlarged side elevational view of the second end of the delivery tube and insulating coating;

FIG. 35 is enlarged side elevational view of the second end of the delivery tube illustrating the tip electrode penetrating tissue;

FIG. 36 is enlarged side elevational view of one of the needle portions with distal electrode and attached wire and insulation;

FIG. 37 is enlarged side elevational view of one of the needle portions with proximal electrode, and openings along the length of the needle and at the needle end; and

FIG. 38 is enlarged side elevational view of one of the needle portions with proximal and distal electrodes, and openings along the length of the needle and at the needle end.

### DETAILED DESCRIPTION OF THE PREFERRED EMBODIMENT

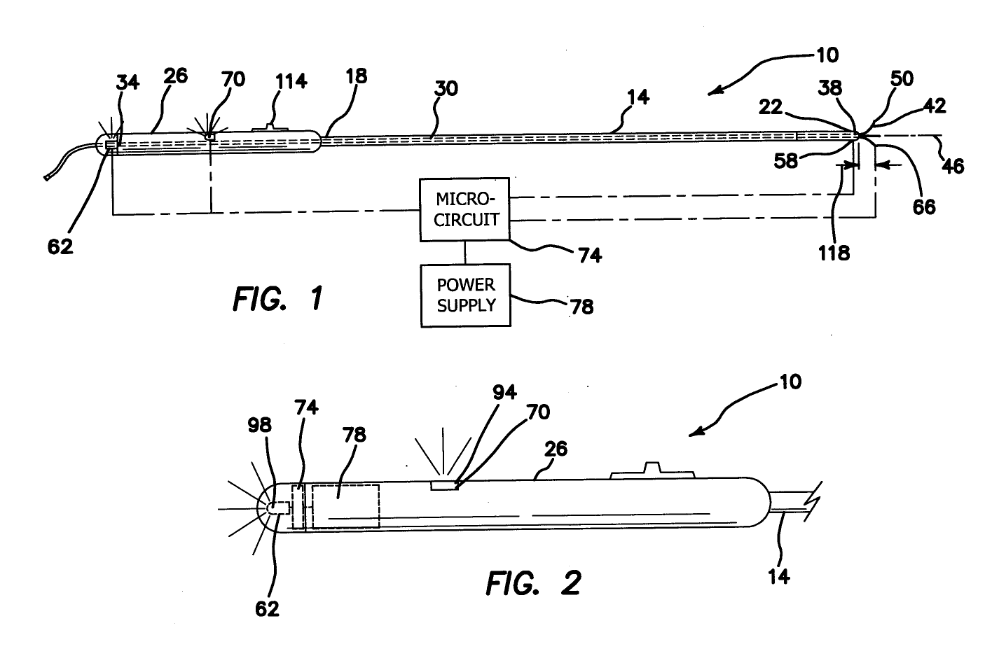

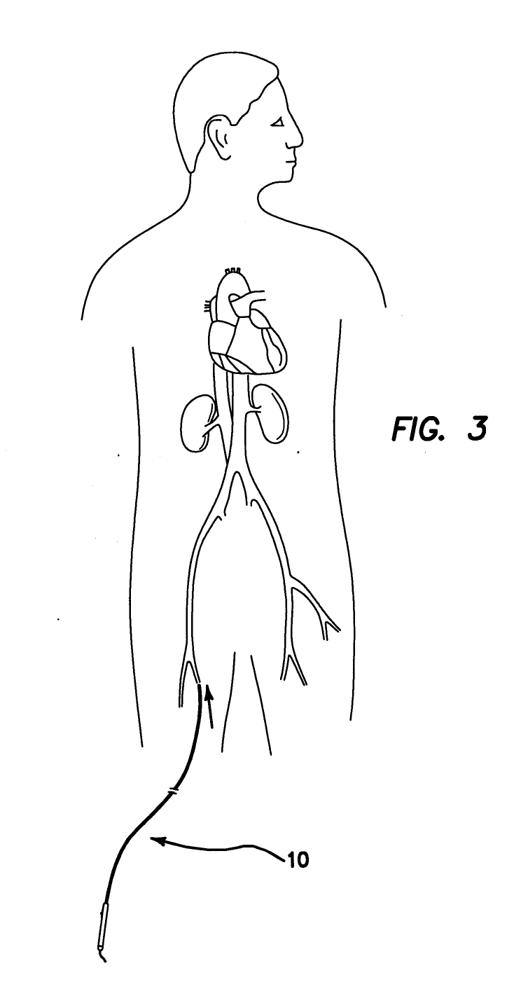

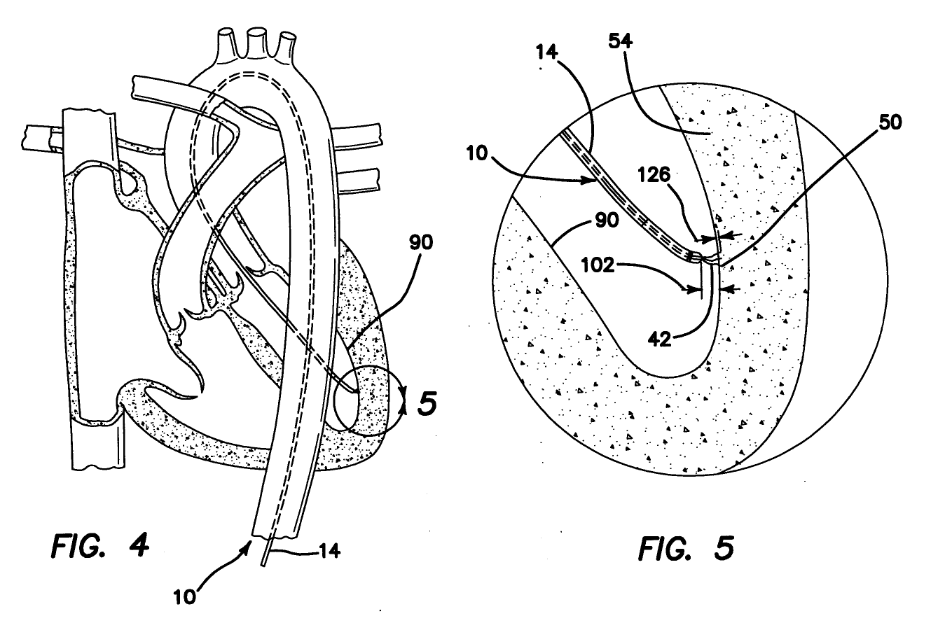

(1) FIG. 1 illustrates a needle injection catheter 10 providing the desired features that may be constructed from the following components. A delivery tube 14 is provided. The delivery tube 14 has a first end 18, a second end 22 and a handle portion 26 adjacent the first end 18. At least one hypotube 30 is provided. The hypotube 30 is sized and shaped to fit slidably within the delivery tube 14. The hypotube has a proximal end 34, a distal end 38 and at least three hollow needle portions 42 connected to and extending outwardly at the distal end 38. The needle portions 42 formed of resilient material, curving outwardly from a central axis 46 of the delivery tube 14 and have ends 50 shaped to penetrate tissue 54 when the hypotube 30 is urged toward the second end 22 of the delivery tube 14. A first sensor 58 is provided. The first sensor 58 is located adjacent the second end 22 of the delivery tube 14 and electrically connected to a first notification device 62 located adjacent the first end 18. At least one second sensor 66 is provided. The second sensor 66 is located adjacent the end 50 of the needle portion 42 and electrically connected to a second notification device 70 located adjacent the first end 18 of the delivery tube 14. A microcircuit 74 is electrically connected to the first 58 and second 66 sensors and to a power supply 78.

(2) In a variant of the invention, as illustrated in FIG. 2, the microcircuit 74 and power supply 78 are contained within the handle portion 26.

(3) In another variant, the first 58 and second 66 sensors detect electrical voltages associated with contact with bodily fluids (not shown), tissues 54 and organ walls 90.

(4) In still another variant, the detected voltages range from 1-30 millivolts.

(5) In yet another variant, the first 58 and second 66 sensors detect temperatures associated with contact with bodily fluids, tissues 54 and organ walls 90.

(6) In a further variant, the first 58 and second 66 sensors detect chemical compositions associated with contact with bodily fluids, tissues 54 and organ walls 90.

(7) In still a further variant, the first 58 and second 66 sensors detect a level of PH associated with contact with bodily fluids, tissues 54 and organ walls 90.

(8) In another variant of the invention, the first 58 and second 66 sensors provide optical imaging to discriminate between bodily fluids, tissues 54 and organ walls 90.

(9) In still another variant, the first 58 and second 66 sensors detect changes in impedance between bodily fluids, tissues 54 and organ walls 90.

(10) In yet another variant, the first 62 and second 70 notification devices are either light emitting devices 94 or sound emitting devices 98.

(11) In a further variant, the second first notification device 62 differs from the first second notification device 70 to indicate a hazard condition should the first sensor 58 contact tissue 54.

(12) In still a further variant, as illustrated in FIG. 5, the needle portions 42 secure the second end 22 of the delivery tube 14 at a predetermined distance 102 from the tissue 54 penetrated by the needle portions 42.

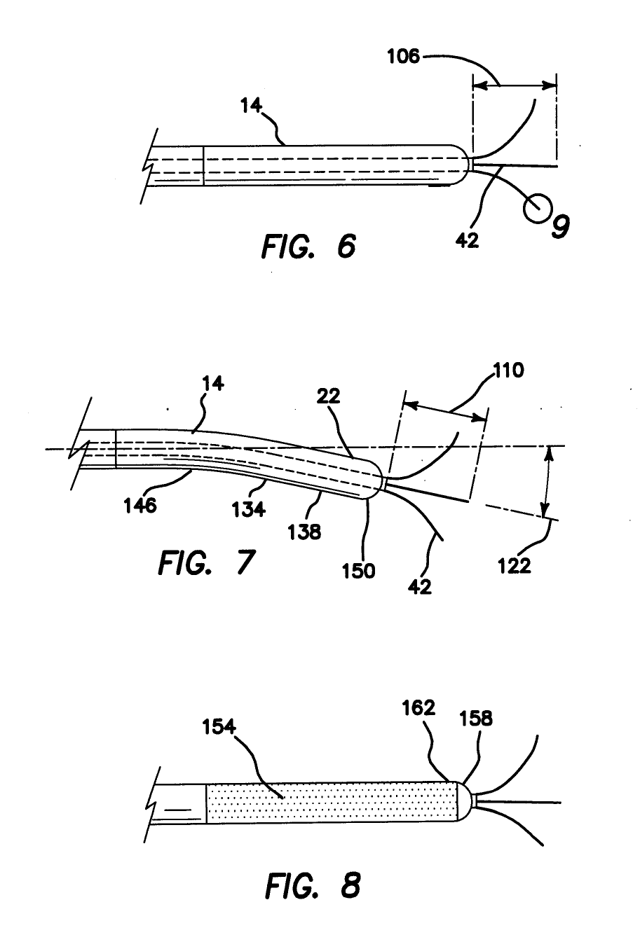

(13) In yet a further variant, as illustrated in FIG. 6, the second end 22 of the delivery tube 14 is secured at a predetermined distance 106 from the tissue 54 during movement due to cardiac cycles.

(14) In still a further variant, as illustrated in FIGS. 1 and 7, extension of the needle portions 42 from the second end 22 of the delivery tube 14 is limited to at least one predetermined distance 110 by an extension control 114 disposed adjacent the handle portion 26.

(15) In another variant of the invention, as illustrated in FIG. 1, the extension control 114 provides for a predetermined minimum extension 118 of the needle portions 42 beyond the second end 22 of the delivery tube 14.

(16) In still another variant, the needle portions 42 extend from 2-7 millimeters beyond the second end 22 of the delivery tube 14.

(17) In yet another variant, as illustrated in FIG. 7, the needle portions 42 are located adjacent the second end 22 of the delivery tube 14 at an angle ranging from 5 to 270 degrees to a central axis 122 of the delivery tube 14. The range varies as the needle portions 42 are extended outwardly from the second end 22 of the delivery tube 14.

(18) In a further variant, as illustrated in FIG. 5, the angular disposition of the needle portions 42 limits a penetration depth 126 for ends 50 of the needle portions 42.

(19) In still a further variant, a lateral movement of the second end 22 of the delivery tube 14 decreases for a specified applied force with extension of the needle portions 42.

(20) In yet a further variant, as illustrated in FIG. 7, a fixed tip 134 is located adjacent the second end 22 of the delivery tube 14. The fixed tip 134 has an angled portion 138 to facilitate perpendicular contact with the surface being injected.

(21) In another variant of the invention, the angled portion 138 is bent at a point 146 spaced 10 mm to 120 mm from a distal end 150 of the fixed tip 134 with an angulation of 10 degrees to 90 degrees.

(22) In still another variant, as illustrated in FIG. 8, the delivery tube 14 further includes a dielectric coated tip 154. The tip 154 has an uncoated section 158 located at a distal end 162 for direct contact with either body tissues 54 or other bodily surfaces.

(23) In yet another variant, the dielectric coated tip 154 includes polyxylylene polymers.

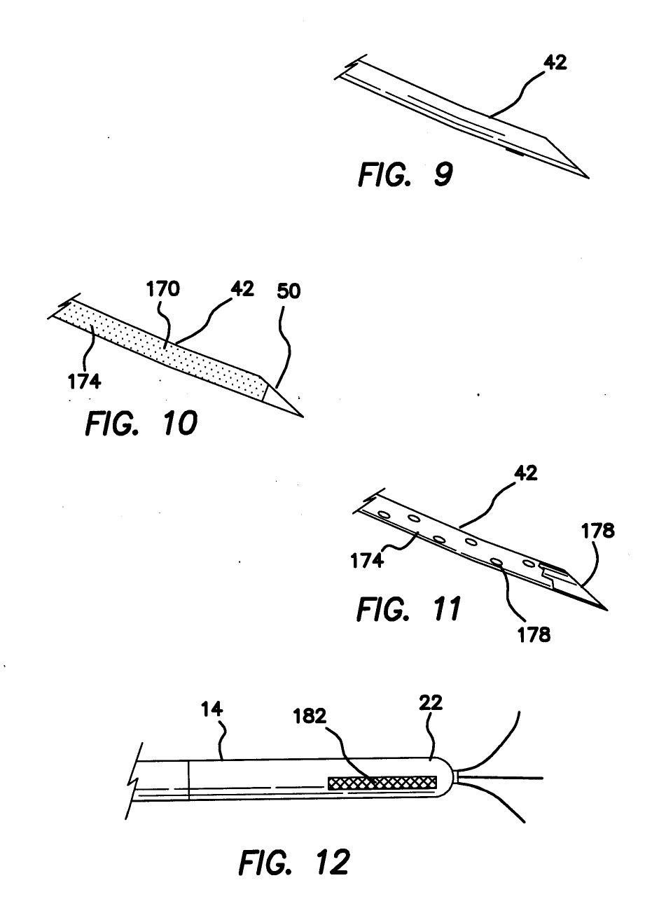

(24) In a further variant, as illustrated in FIG. 10, the needle portions 42 are dielectric coated 170 along their exterior length 174 and not dielectric coated 170 at their ends 50.

(25) In still a further variant, as illustrated in FIG. 11, the needle portions 42 include openings 178 at their ends 50 and along their exterior length 174.

(26) In yet a further variant, as illustrated in FIG. 12, the second end 22 of the delivery tube 14 includes a magnetic element 182.

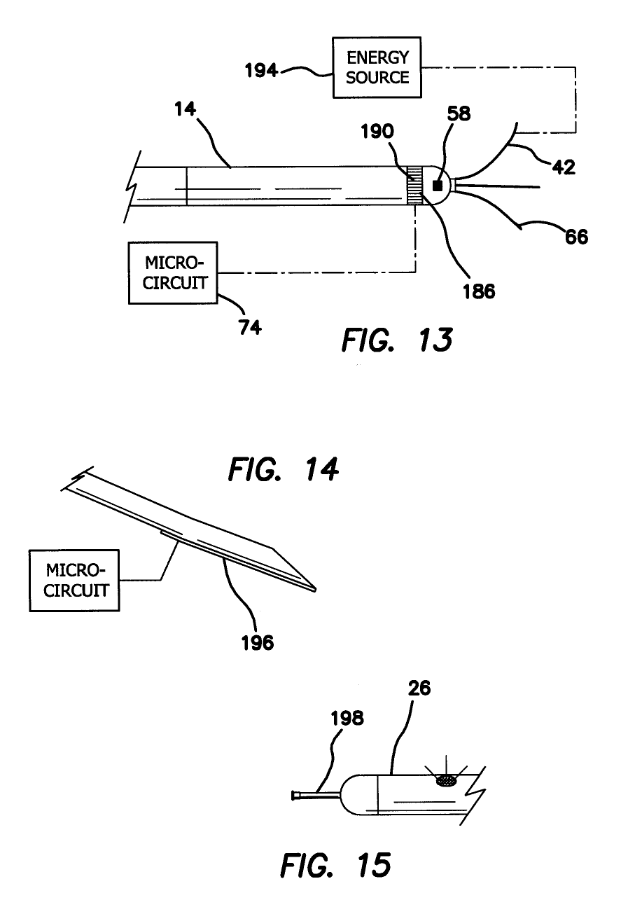

(27) In another variant of the invention, as illustrated in FIG. 13, the delivery tube 14 includes at least one ring electrode 186.

(28) In still another variant, the ring electrode 186 is either a voltage or impedance reference 190.

(29) In yet another variant, the reference 190 is electrically connected to the microcircuit 74.

(30) In a further variant, the microcircuit 74 computes impedance from an alternating current between the reference 190 and the second sensor 66.

(31) In still a further variant, the microcircuit 74 computes impedance from an alternating current between the reference 190 and the first sensor 58.

(32) In yet a further variant, an energy source 194 is provided. The energy source 194 is connected to at least one of the needle portions 42.

(33) In another variant of the invention, the external energy source 194 is selected from the group consisting of radio frequency (RF) energy and laser energy.

(34) In still another variant, as illustrated in FIG. 14, at least one of the needle portions 42 is a thermocouple 196 for measuring temperature at a location of the needle portions 42.

(35) In yet another variant, the needle portions 42 are formed of material selected from the group consisting of metallic material, shape memory metal, duromers, fiber reinforced materials, polymer and polymer material with a highly refractive index capable of transmitting and receiving an optical signal.

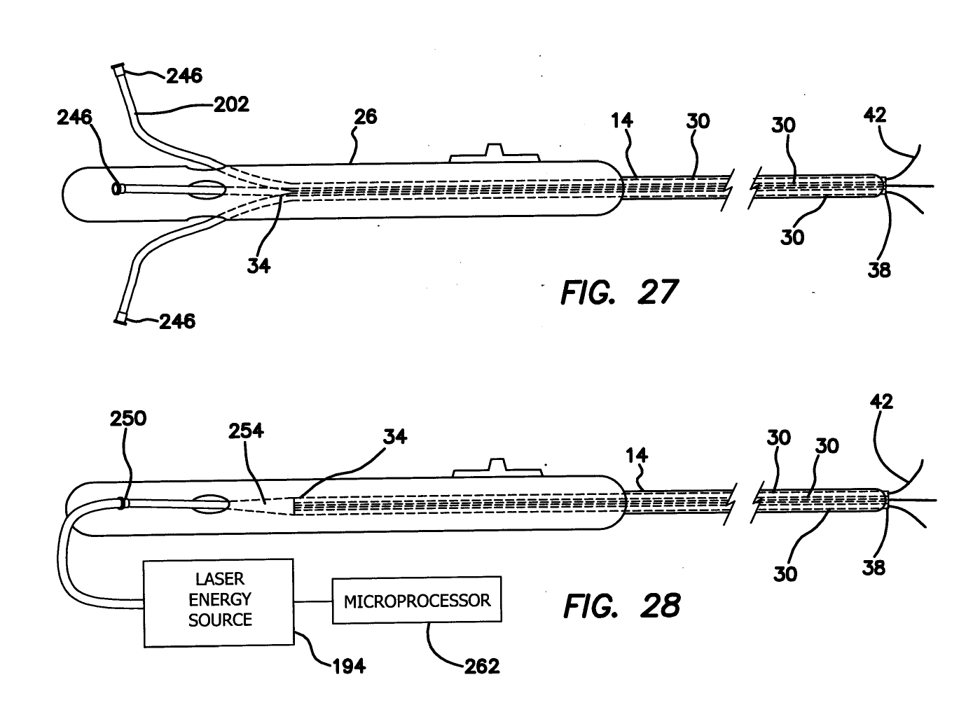

(36) In a further variant, as illustrated in FIGS. 15 and 27, the hypotube 30 is connected to either a fixed luer connection 198 or a flexible luer connection 202 adjacent the handle portion 26 to facilitate introduction of an injectate.

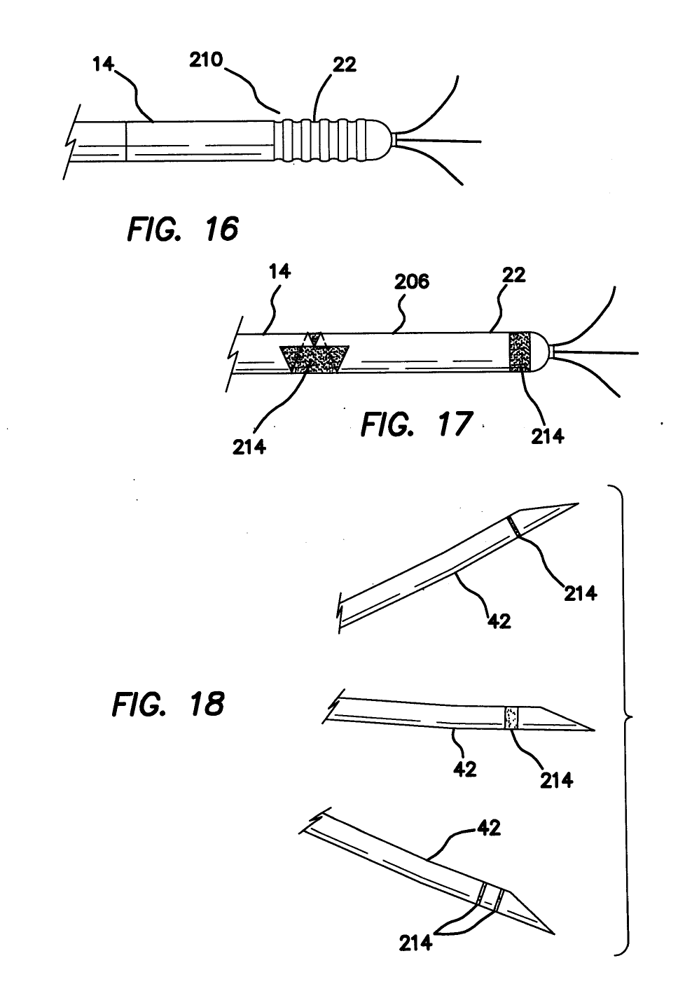

(37) In still a further variant, as illustrated in FIGS. 16 and 17, either a fixed 206 or deflectable 210 tip is located adjacent the second end 22 of the delivery tube 14.

(38) In yet a further variant, as illustrated in FIG. 17, at least one radiopaque marker 214 is located either upon or within the delivery tube 14.

(39) In another variant of the invention, the radiopaque marker 214 is located adjacent the second end 22 of the delivery tube 14.

(40) In still another variant, the radiopaque markers 214 are differentiated so that the location of each marker 214 relative to other markers 214 and the second end 22 of the delivery tube 14 is determined.

(41) In yet another variant, the radiopaque markers 214 are located circumferentially either upon or within the delivery tube 14 and are tapered in form to indicate the radial position of the delivery tube when viewed with a radiological scanning system (not shown).

(42) In a further variant, as illustrated in FIG. 18, at least one radiopaque marker 214 is located adjacent the end 50 of at least one of the needle portions 42.

(43) In still a further variant, the radiopaque markers 214 are differentiated for each of the needle portions 42, thereby permitting an operator to identify a location of each needle portion 42.

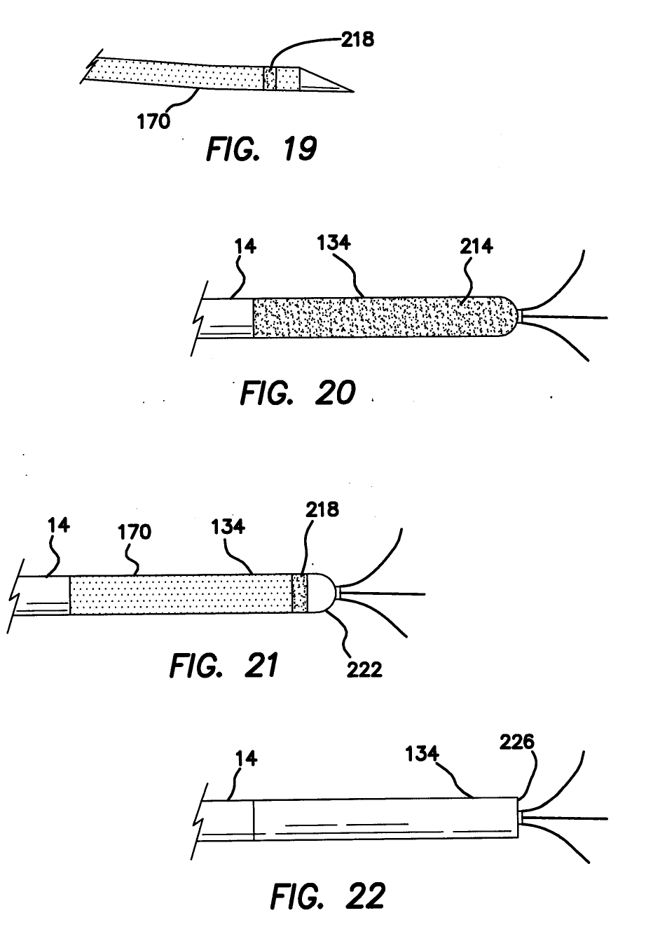

(44) In yet a further variant, as illustrated in FIG. 19, the needle portions 42 includes a nano-particle enhanced radiopaque coating 218.

(45) In another variant of the invention, the needle portions 42 include a nano-particle enhanced radiopaque coating 218 mixed with a dielectrtic coating 170.

(46) In still another variant, any of the first 58 and second 66 sensors are radiopaque markers 214.

(47) In yet another variant, as illustrated in FIG. 20, the tip 134 is a radiopaque marker 214.

(48) In a further variant, as illustrated in FIG. 21, the tip 134 includes a nano-particle enhanced radiopaque coating 218.

(49) In still a further variant, the tip 134 includes a nano-particle enhanced radiopaque coating 218 mixed with a dielectrtic coating 170.

(50) In yet a further variant, as illustrated in FIGS. 21 and 22, the tip 134 is rounded 222 or flat 226 for use in ablation.

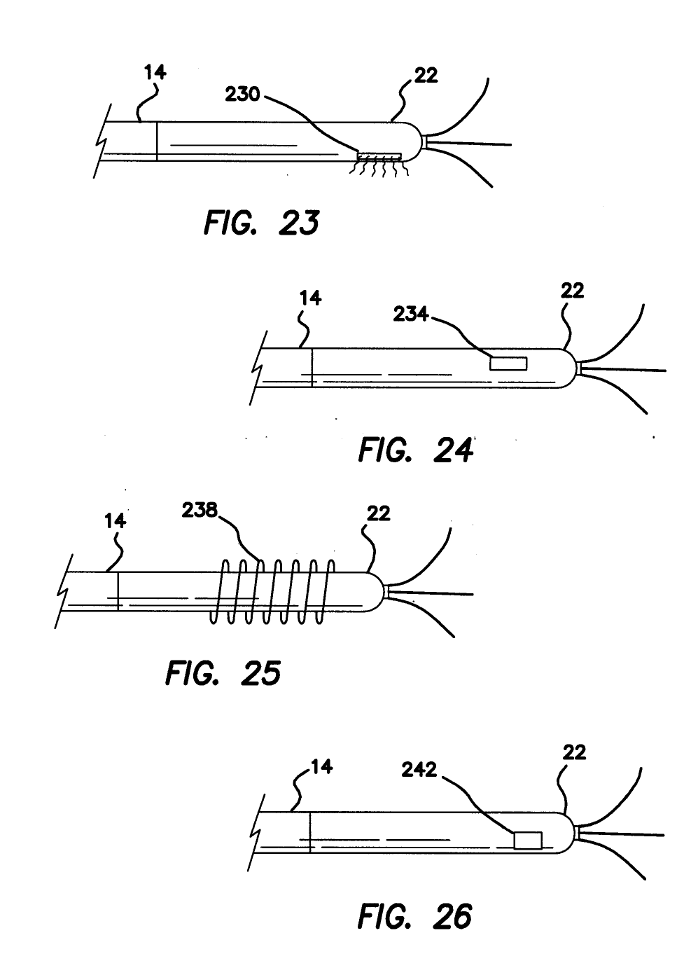

(51) In another variant of the invention, as illustrated in FIG. 23, the second end 22 of the delivery tube 14 includes an ultrasound transducer 230.

(52) In still another variant, as illustrated in FIG. 24, the second end 22 of the delivery tube 14 includes an RF transmitter 234.

(53) In yet another variant, as illustrated in FIG. 25, the second end 22 of the delivery tube 14 includes an electromagnetic coil 238.

(54) In a further variant, as illustrated in FIG. 26, the second end 22 of the delivery tube 14 includes a transponder 242.

(55) In still a further variant, as illustrated in FIG. 1, the delivery tube 14 encloses a single hypotube 30. The single hypotube 30 is divided into at least three communicating needle portions 42 at the distal end 38.

(56) In yet a further variant, as illustrated in FIG. 27, the delivery tube 14 encloses at least three hypotubes 30. Each of the hypotubes 30 has a needle portion 42 at the distal end 38 and a separate connection point 246 at the proximal end 34.

(57) In another variant of the invention, as illustrated in FIG. 28, the delivery tube 14 encloses at least three hypotubes 30. Each of the hypotubes 30 has a needle portion 42 at the distal end 38 and joins into a common connection point 250 at the proximal end 34.

(58) In still another variant, the hypotube 30 is formed of polymer material with a highly refractive index 254 capable of transmitting and receiving an optical signal.

(59) In yet another variant, the hypotube 30 is connected to a source of laser energy 194 and a microprocessor 262.

(60) In another variant of the invention, the needle portions 42 include openings 266 at their ends 50 and along their exterior length 270. A first insulating coating 274 is provided. The first insulating coating 274 extends from the proximal end (not shown) to a point 282 adjacent the end 50 of the needle portion 42. A first conducting coating 286 is provided. The first conducting coating 286 extends from the proximal end to a point adjacent a distal end 294 of the first insulating coating 274. A second insulating coating 290 is provided. The second insulating coating 290 extends from the proximal end to a point adjacent the opening 266 along the exterior length 270 of the needle portion 42. A second conducting coating 302 is provided. The second conducting coating 302 extends from the proximal end to a point adjacent the opening 266 along the exterior length 270 of the needle portion 42. A third insulating coating 310 is provided. The third insulating coating 310 extends from the proximal end to a point adjacent a distal end 306 of second conducting coating 302. Each of the insulating 274, 290, 310 and conducting 286, 302 coatings has an aperture (not shown) sized and shaped to surround the opening 266 along the exterior length 270 of the needle portion 42. The first 286 and second 302 conducting coatings are connected to the power supply 78, a pulse generator 324 and a pulse generation switch 326. When a current is introduced to the conducting coatings 286, 302, a magnetic field is established adjacent the openings 266, at the ends 50 and along the exterior length 270 of the needle portion 42.

(61) In still a further variant, as illustrated in FIG. 30, a needle injection catheter 10, includes a delivery tube 14. The delivery tube 14 has a first end 18, a second end 22 and a handle portion 26 adjacent the first end 18. At least one hypotube 30 is provided. The hypotube 30 is sized and shaped to fit slidably within the delivery tube 14. The hypotube 30 has a proximal end 34, a distal end 38 and at least three hollow needle portions 42 connected to and extending outwardly at the distal end 38. The needle portions 42 are formed of resilient material, curving outwardly from a central axis 46 of the delivery tube 14 and have ends 50 shaped to penetrate tissue 54 when the hypotube 30 is urged toward the second end 22 of the delivery tube. At least one reference electrode 338 is provided. The reference electrode 338 is located on the delivery tube 14, spaced from the second end 22. At least one proximal electrode 342 is provided. The proximal electrode 342 is located adjacent and spaced from the end 50 of the needle portion 42. The proximal electrode 342 is electrically connected to a first notification device 62 located adjacent the first end 18 of the delivery tube 14. A microcircuit 74 is electrically connected to the proximal electrode 342, the reference electrode 338 and to a power supply 78.

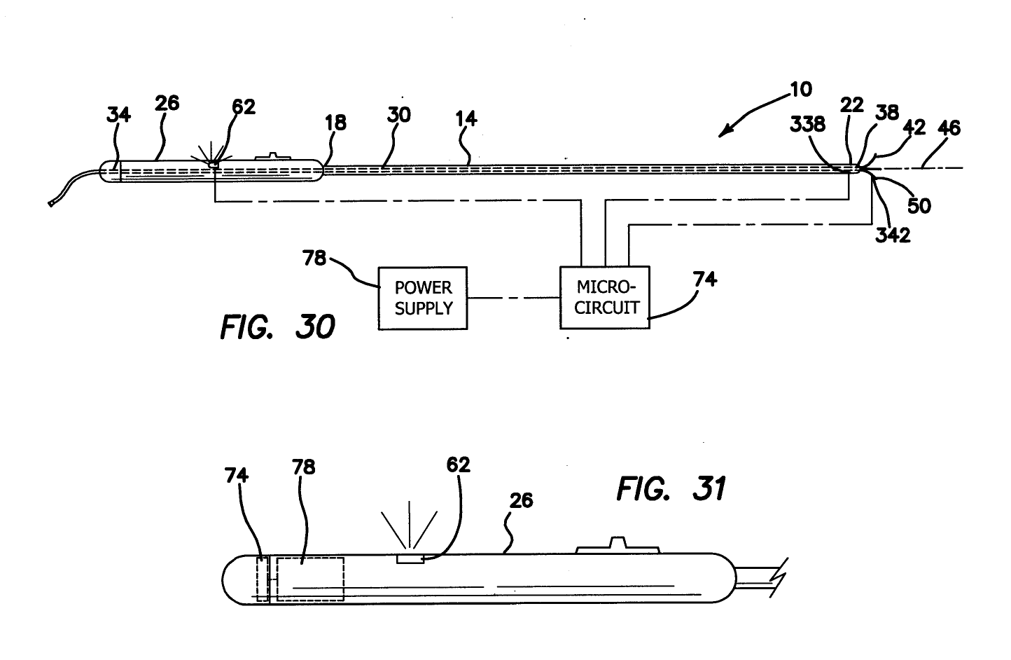

(62) In yet a further variant, as illustrated in FIG. 31, the microcircuit 74 and the power supply 78 are contained within the handle portion 26.

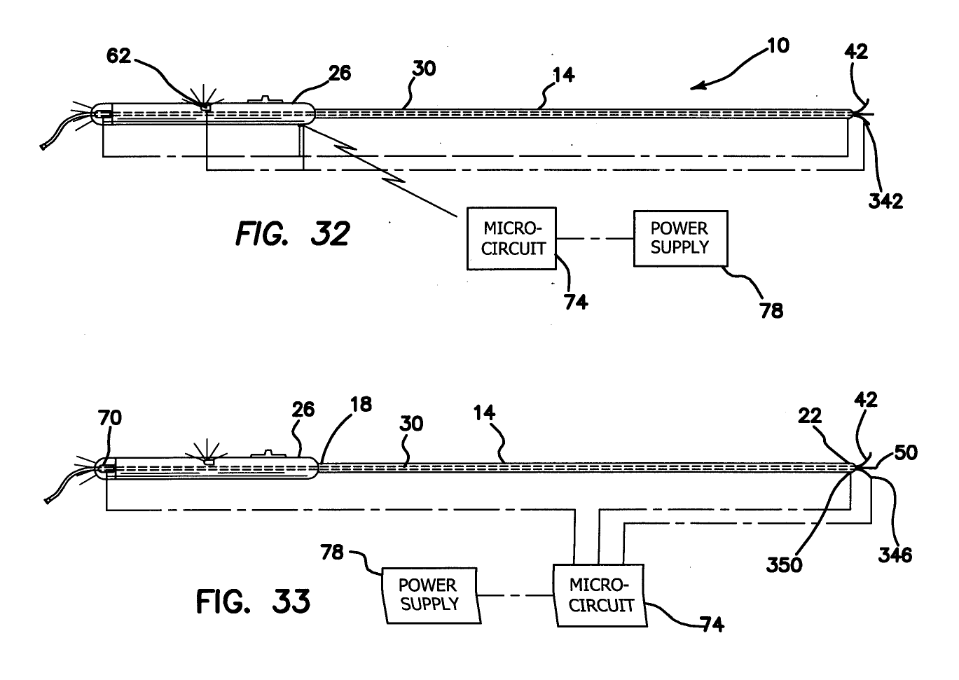

(63) In another variant of the invention, as illustrated in FIG. 32, the microcircuit 74 and the power supply 78 are wirelessly connected to the catheter 10.

(64) In still another variant, the microcircuit 74 is both analog and digital.

(65) In yet another variant, as illustrated in FIG. 33, at least one distal electrode 346 is provided. The distal electrode 346 is located adjacent the end 50 of the needle portion 42 and electrically connected to the microcircuit 74.

(66) In a further variant, a tip electrode 350 is provided. The tip electrode 350 is located adjacent the second end 22 of the delivery tube 14 and electrically connected to a second notification device 70 located adjacent the first end 18 of the delivery tube 14 and connected to the microcircuit 74.

(67) In still a further variant, as illustrated in FIG. 30, the at least one reference electrode 338 is detected by impedance based mapping systems (not shown).

(68) In yet a further variant, the reference electrode 338 is formed of a mixture of platinum and iridium.

(69) In another variant of the invention, impedance is measured between 1 KHz and 75 KHz.

(70) In still another variant, voltage is measured between 1-50 millivolts.

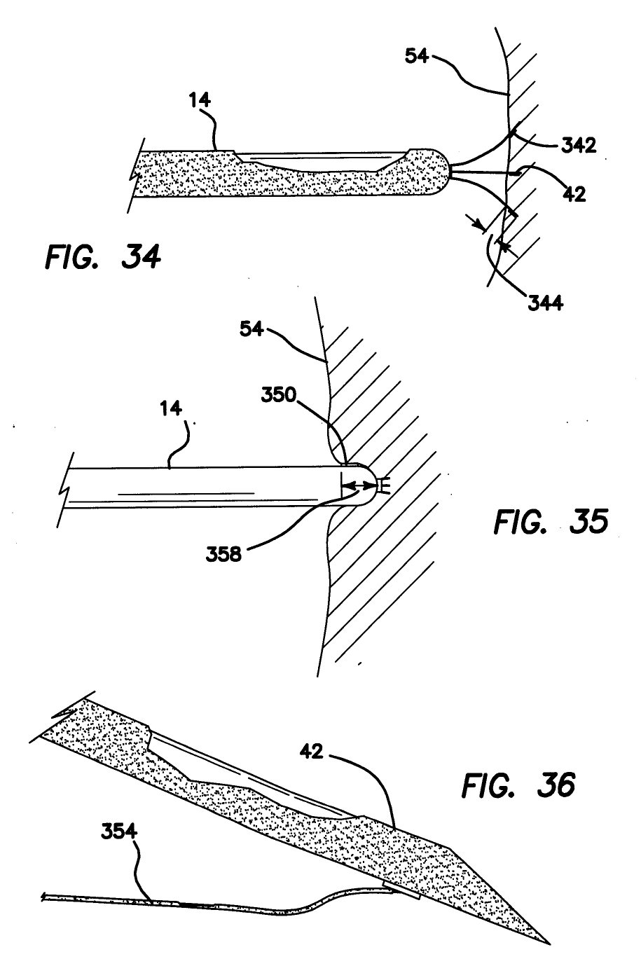

(71) In yet another variant, as illustrated in FIG. 34, the delivery tube 14 is insulated.

(72) In a further variant, as illustrated in FIG. 36, the needle portions 42 are insulated with polyxylylene polymers.

(73) In still a further variant, the needle portions 42 are coated with 1-40 microns of insulation.

(74) In yet a further variant, wires 354 connected to the needle portions 42 are insulated with polyxylylene polymers.

(75) In another variant of the invention, as illustrated in FIG. 34, the proximal electrode 342 is spaced from the end of the needle portion 42 by a first predetermined distance 344 for entry of the needle portion 42 into tissue 54.

(76) In still another variant, the first notification device 62 is activated only when all three of the needle portions 42 have the proximal electrode 342 in contact with tissue 54.

(77) In yet another variant, as illustrated in FIG. 35, the second notification device 70 is activated only when the tip electrode 350 is embedded is tissue 54 for a first predetermined depth 358.

(78) In a further variant, the first predetermined depth 358 is 0.5-4 mm.

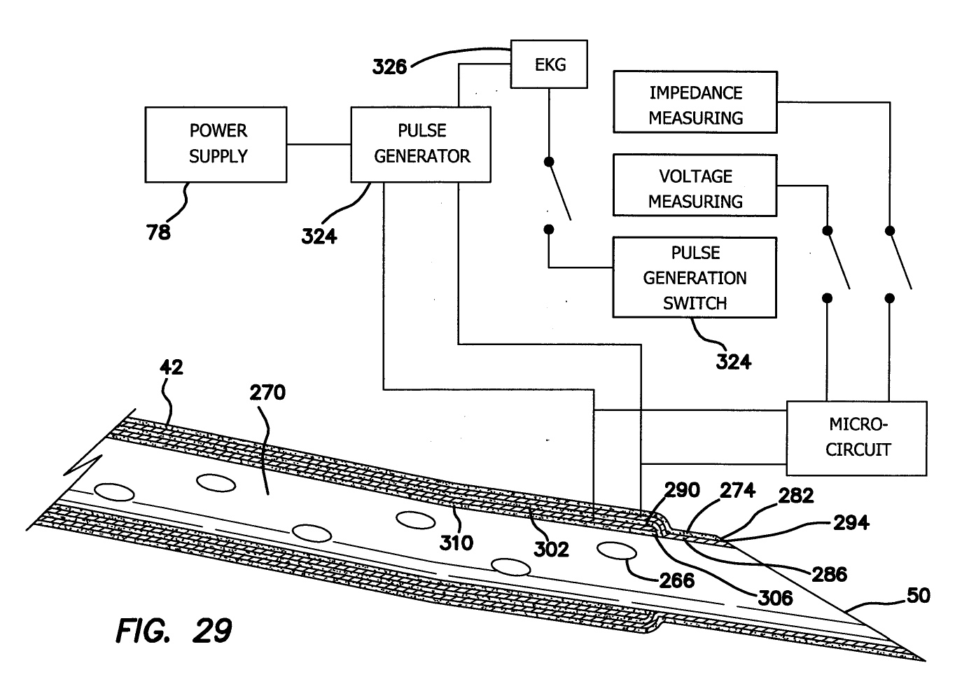

(79) In still a further variant, as illustrated in FIG. 29, the needle injection catheter 10 includes a pulse generator 324. The pulse generator 324 is electrically connected to a switch 326. The switch 326 selects between impedance measuring, voltage measuring, and pulse generation.

(80) In yet a further variant, the pulse generator 324 produces a pulse ranging from 1-700 volts for 100 microseconds to 10 milliseconds at up to 200 mA.

(81) In another variant of the invention, as illustrated in FIGS. 32 and 33, the pulse generator 324 creates an electrical field between the proximal electrode 342 and the distal electrode 346 on each of the needle portions 42.

(82) In still another variant, the pulse generator 324 creates an electrical field between the proximal electrode 342 and the distal electrode 346 on each of the needle portions 42 successively.

(83) In yet another variant, as illustrated in FIG. 29, the pulse generator 324 is gated to an electrocardiogram (EKG) device 326 to prevent pulse generation during vulnerable portions of cardiac cycles.

(84) In a further variant, as illustrated in FIGS. 32 and 33, impedance between the proximal electrode 342 and the distal electrode 346 is simultaneously measured for each of the needle portions 42.

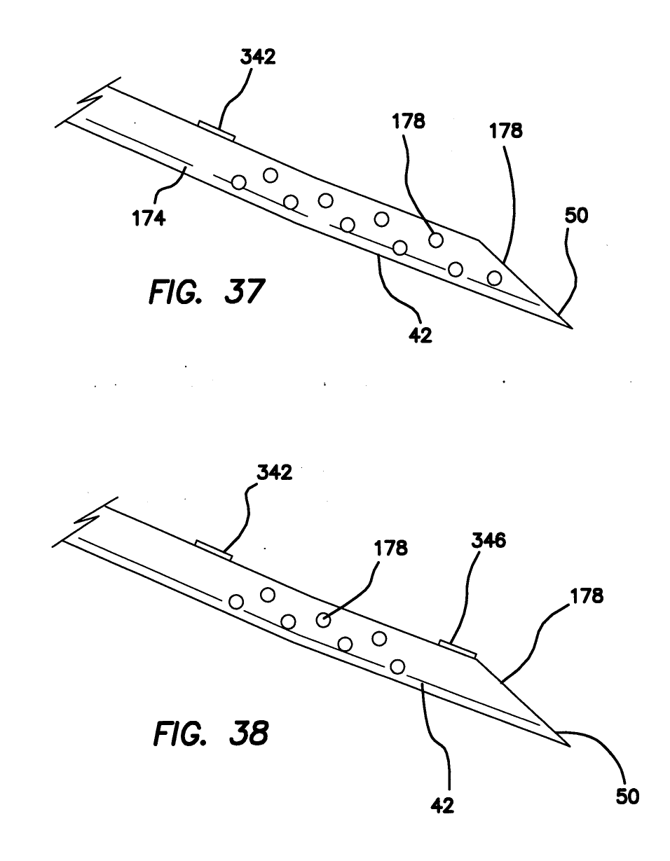

(85) In still a further variant, as illustrated in FIG. 37, the needle portions 42 include openings 178 along their exterior length 174. The openings 178 extend from the proximal electrode 342 toward the end 50 of the needle portion 42.

(86) In yet a further variant, as illustrated in FIG. 38, the needle portions 42 include openings 178 along their exterior length 174, the openings 178 extending from the proximal electrode 342 to the distal electrode 346.

(87) In another variant, each of the needle portions 42 has an additional opening 178 at the end 50 of the needle portion 42.

(88) In a final variant, each of the needle portions 42 has an additional opening 178 at the end 50 of the needle portion 42.

The needle injection catheter 10 has been described with reference to particular embodiments. Other modifications and enhancements can be made without departing from the spirit and scope of the claims that follow.

1. A needle injection catheter, comprising:
a delivery tube, said delivery tube having a first end, a second end and a handle portion adjacent said first end;
at least one hypotube, said hypotube being sized and shaped to fit slidably within said delivery tube, having a proximal end, a distal end and at least three hollow needle portions connected to and extending outwardly at said distal end;
said needle portions, being formed of resilient material, curving outwardly from a central axis of said delivery tube and having ends shaped to penetrate tissue when said hypotube is urged toward said second end of said delivery tube;
a first sensor, said first sensor being disposed adjacent said second end of said delivery tube and electrically connected to a first notification device disposed adjacent said first end; and
at least one second sensor, said second sensor being disposed adjacent said end of said needle portion and electrically connected to a second notification device disposed adjacent said first end of said delivery tube; and
a microcircuit electrically connected to said first and second sensors and to a power supply.

2. The needle injection catheter, as described in claim 1, wherein said microcircuit and power supply are contained within said handle portion.

3. The needle injection catheter, as described in claim 1, wherein said first and second sensors detect electrical voltages associated with contact with bodily fluids, tissues and organ walls.

4. The needle injection catheter, as described in claim 3, wherein said detected voltages range from 1-30 millivolts.

5. The needle injection catheter, as described in claim 1, wherein said first and second sensors detect temperatures associated with contact with bodily fluids, tissues and organ walls.

6. The needle injection catheter, as described in claim 1, wherein said first and second sensors detect chemical compositions associated with contact with bodily fluids, tissues and organ walls.

7. The needle injection catheter, as described in claim 1, wherein said first and second sensors detect a level of PH associated with contact with bodily fluids, tissues and organ walls.

8. The needle injection catheter, as described in claim 1, wherein said first and second sensors provide optical imaging to discriminate between bodily fluids, tissues and organ walls.

9. The needle injection catheter, as described in claim 1, wherein said first and second sensors detect changes in impedance between bodily fluids, tissues and organ walls.

10. The needle injection catheter, as described in claim 1, wherein said first and second notification devices are either of light emitting devices and sound emitting devices.

11. The needle injection catheter, as described in claim 1, wherein said second notification device differs from said first notification device to indicate a hazard condition should said first sensor contact tissue.

12. The needle injection catheter, as described in claim 1, wherein said needle portions secure said second end of said delivery tube at a predetermined distance from said tissue penetrated by said needle portions.

13. The needle injection catheter, as described in claim 12, wherein said second end of said delivery tube is secured at a predetermined distance from said tissue during movement due to cardiac cycles.

14. The needle injection catheter, as described in claim 1, wherein extension of said needle portions from said second end of said delivery tube is limited to at least one predetermined distance by an extension control disposed adjacent said handle portion.

15. The needle injection catheter, as described in claim 14, wherein said extension control provides for a predetermined minimum extension of said needle portions beyond said second end of said delivery tube.

16. The needle injection catheter, as described in claim 1, wherein said needle portions extend from 2-7 millimeters beyond said second end of said delivery tube.

17. The needle injection catheter, as described in claim 1, wherein said needle portions are disposed adjacent said second end of said delivery tube at an angle ranging from 5 to 270 degrees to a central axis of said delivery tube, said range varying as said needle portions are extended outwardly from said second end of said delivery tube.

18. The needle injection catheter, as described in claim 17, wherein said angular disposition of said needle portions limits a penetration depth for distal ends of said needle portions.

19. The needle injection catheter, as described in claim 1, wherein a lateral movement of said second end of said delivery tube decreases for a specified applied force with extension of said needle portions.

20. The needle injection catheter, as described in claim 1, wherein a fixed tip is disposed adjacent said second end of said delivery tube, said fixed tip having an angled portion to facilitate perpendicular contact with the surface being injected.

21. The needle injection catheter, as described in claim 20, wherein said angled portion is bent at a point spaced 10 mm to 120 mm from a distal end of the fixed tip with an angulation of 10° to 90°.

22. The needle injection catheter, as described in claim 1, wherein said delivery tube further comprises a dielectric coated tip, said tip having an uncoated section disposed at a distal end for direct contact with either of body tissues and other bodily surfaces.

23. The needle injection catheter, as described in claim 21, wherein said dielectric coated tip comprises polyxylylene polymers.

24. The needle injection catheter, as described in claim 1, wherein said needle portions are dielectric coated along their exterior length and not dielectric coated at their distal ends.

25. The needle injection catheter, as described in claim 1, wherein said needle portions comprise openings at their distal ends and along their exterior length.

26. The needle injection catheter, as described in claim 1, wherein said second end of said delivery tube comprises a magnetic element.

27. The needle injection catheter, as described in claim 1, wherein said delivery tube comprises at least one ring electrode.

28. The needle injection catheter, as described in claim 26, wherein said ring electrode is either of a voltage and impedance reference.

29. The needle injection catheter, as described in claim 27, wherein said reference is electrically connected to said microcircuit.

30. The needle injection catheter, as described in claim 28, wherein said microcircuit computes impedance from an alternating current between said reference and said second sensor.

31. The needle injection catheter, as described in claim 28, wherein said microcircuit computes impedance from an alternating current between said reference and said first sensor.

32. The needle injection catheter, as described in claim 1, further comprising an external energy source, said energy source connected to at least one of said needle portions.

33. The needle injection catheter, as described in claim 31, wherein said external energy source is selected from the group consisting of:
radio frequency (RF) energy and laser energy.

34. The needle injection catheter, as described in claim 1, wherein at least one of said needle portions is a thermocouple for measuring temperature at a location of said needle portions.

35. The needle injection catheter, as described in claim 1, wherein said needle portions are formed of material selected from the group consisting of:
metallic material, shape memory metal, duromers, fiber reinforced materials, polymer and polymer material with a highly refractive index capable of transmitting and receiving an optical signal.

36. The needle injection catheter, as described in claim 1, wherein said hypotube is connected to either of a fixed luer connection and a flexible luer connection adjacent said handle portion to facilitate introduction of an injectate.

37. The needle injection catheter, as described in claim 1, wherein either of a fixed and deflectable tip is disposed adjacent said second end of said delivery tube.

38. The needle injection catheter, as described in claim 1, further comprising at least one radiopaque marker disposed either of upon and within said delivery tube.

39. The needle injection catheter, as described in claim 37, wherein said radiopaque marker is disposed adjacent said second end of said delivery tube.

40. The needle injection catheter, as described in claim 37, wherein said radiopaque markers are differentiated so that the location of each marker relative to other markers and said second end of said delivery tube is determined.

41. The needle injection catheter, as described in claim 37, wherein said radiopaque marker is disposed circumferentially either of upon and within said delivery tube and is tapered in form to indicate the radial position of said delivery tube when viewed with a radiological scanning system.

42. The needle injection catheter, as described in claim 1, further comprising at least one radiopaque marker disposed adjacent said end of at least one of said needle portions.

43. The needle injection catheter, as described in claim 41, wherein said radiopaque markers are differentiated for each of said needle portions, thereby permitting an operator to identify a location of each needle portion.

44. The needle injection catheter, as described in claim 1, wherein said needle portions comprise a nano-particle enhanced radiopaque coating.

45. The needle injection catheter, as described in claim 1, wherein said needle portions comprise a nano-particle enhanced radiopaque coating mixed with a dielectrtic coating.

46. The needle injection catheter, as described in claim 1, wherein any of said first and second sensors are radiopaque markers.

47. The needle injection catheter, as described in claim 36, wherein said tip is a radiopaque marker.

48. The needle injection catheter, as described in claim 36, wherein said tip comprises a nano-particle enhanced radiopaque coating.

49. The needle injection catheter, as described in claim 36, wherein said tip comprises a nano-particle enhanced radiopaque coating mixed with a dielectrtic coating.

50. The needle injection catheter, as described in claim 36, wherein said tip is either of rounded and flat for use in ablation.

51. The needle injection catheter, as described in claim 1, wherein said second end of said delivery tube comprises an ultrasound transducer.

52. The needle injection catheter, as described in claim 1, wherein said second end of said delivery tube comprises an RF transmitter.

53. The needle injection catheter, as described in claim 1, wherein said second end of said delivery tube comprises an electromagnetic coil.

54. The needle injection catheter, as described in claim 1, wherein said second end of said delivery tube comprises a transponder.

55. The needle injection catheter, as described in claim 1, wherein said delivery tube encloses a single hypotube, said single hypotube divided into at least three communicating needle portions at said distal end.

56. The needle injection catheter, as described in claim 1, wherein said delivery tube encloses at least three hypotubes, each of said hypotubes having a needle portion at said distal end and a separate connection point at said proximal end.

57. The needle injection catheter, as described in claim 1, wherein said delivery tube encloses at least three hypotubes, each of said hypotubes having a needle portion at said distal end and joining into a common connection point at said proximal end.

58. The needle injection catheter, as described in claim 1, wherein said hypotube is formed of polymer material with a highly refractive index capable of transmitting and receiving an optical signal.

59. The needle injection catheter, as described in claim 58, wherein said hypotube is connected to a source of laser energy and a microprocessor.

60. The needle injection catheter, as described in claim 56, wherein:
said needle portions comprise openings at their distal ends and along their exterior length
a first insulating coating, said first insulating coating extending from said proximal end to a point adjacent a distal end of said needle portion;
a first conducting coating, said first conducting coating extending from said proximal end to a point adjacent a distal end of said first insulating coating;
a second insulating coating, said second insulating coating extending from said proximal end to a point adjacent a distal end of said first conducting coating;
a second conducting coating, said second conducting coating extending from said proximal end to a point adjacent said opening along said exterior length of said needle portion;
a third insulating coating, said third insulating coating extending from said proximal end to a point adjacent a distal end of second conducting coating;
each of said insulating and conducting coatings having an aperture sized and shaped to surround said opening along said exterior length of said needle portion;
said first and second conducting coatings connected to said power supply and a pulsing switch; and
whereby, when a current is introduced to said conducting coatings, a magnetic field is established adjacent said openings at said distal ends and along said exterior length of said needle portion.

61. A needle injection catheter, comprising:
a delivery tube, said delivery tube having a first end, a second end and a handle portion adjacent said first end;
at least one hypotube, said hypotube being sized and shaped to fit slidably within said delivery tube, having a proximal end, a distal end and at least three hollow needle portions connected to and extending outwardly at said distal end;
said needle portions, being formed of resilient material, curving outwardly from a central axis of said delivery tube and having ends shaped to penetrate tissue when said hypotube is urged toward said second end of said delivery tube;
at least one reference electrode, said reference electrode being disposed upon said delivery tube, spaced from said second end;
at least one proximal electrode, said proximal electrode being disposed adjacent and spaced from said end of said needle portion and electrically connected to a first notification device disposed adjacent said first end of said delivery tube; and
a microcircuit electrically connected to said proximal electrode, said reference electrode and to a power supply.

62. The needle injection catheter as described in claim 61, wherein said microcircuit and said power supply are contained within said handle portion.

63. The needle injection catheter as described in claim 61, wherein said microcircuit and said power supply are wirelessly connected to said catheter.

64. The needle injection catheter as described in claim 61, wherein said microcircuit is both analog and digital.

65. The needle injection catheter, as described in claim 61, further comprising at least one distal electrode, said distal electrode being disposed adjacent said end of said needle portion and electrically connected to said microcircuit.

66. The needle injection catheter, as described in claim 61, further comprising a tip electrode, said tip electrode being disposed adjacent said second end of said delivery tube and electrically connected to a second notification device disposed adjacent said first end of said delivery tube and connected to said microcircuit.

67. The needle injection catheter, as described in claim 61, wherein said at least one reference electrode is detected by impedance based mapping systems.

68. The needle injection catheter, as described in claim 61, wherein said reference electrode is formed of a mixture of platinum and iridium,

69. The needle injection catheter, as described in claim 61, wherein impedance is measured between 1 KHz and 75 KHz.

70. The needle injection catheter, as described in claim 61, wherein voltage is measured between 1-50 millivolts.

71. The needle injection catheter, as described in claim 61, wherein said delivery tube is insulated.

72. The needle injection catheter, as described in claim 61, wherein said needle portions are insulated with polyxylylene polymers.

73. The needle injection catheter, as described in claim 72, wherein said needle portions are coated with 1-40 microns of insulation.

74. The needle injection catheter, as described in claim 61, wherein wires connected to said needle portions are insulated with polyxylylene polymers.

75. The needle injection catheter, as described in claim 61, wherein said proximal electrode is spaced from said end of said needle portion by a first predetermined distance for entry of said needle portion into tissue.

76. The needle injection catheter, as described in claim 61, wherein said first notification device is activated only when all three of said needle portions have said proximal electrode in contact with tissue.

77. The needle injection catheter, as described in claim 63, wherein said second notification device is activated only when said tip electrode is embedded is tissue for a first predetermined depth.

78. The needle injection catheter, as described in claim 77, wherein said first predetermined depth is 0.5-4 mm.

79. The needle injection catheter, as described in claim 65, further comprising:
a pulse generator, said pulse generator electrically connected to a switch;
said switch selecting between impedance measuring, voltage measuring, and pulse generation.

80. The needle injection catheter, as described in claim 79, wherein said pulse generator produces a pulse ranging from 1-700 volts for 100 microseconds to 10 milliseconds at up to 200 mA.

81. The needle injection catheter, as described in claim 79, wherein said pulse generator creates an electrical field between said proximal electrode and said distal electrode on each of said needle portions.

82. The needle injection catheter, as described in claim 79, wherein said pulse generator creates an electrical field between said proximal electrode and said distal electrode on each of said needle portions successively.

83. The needle injection catheter, as described in claim 79, wherein said pulse generator is gated to an electrocardiogram (EKG) device to prevent pulse generation during vulnerable portions of cardiac cycles.

84. The needle injection catheter, as described in claim 65, wherein impedance between said proximal electrode and said distal electrode is simultaneously measured for each of said needle portions.

85. The needle injection catheter, as described in claim 61, wherein said needle portions comprise openings along their exterior length, said openings extending from said proximal electrode toward said end of said needle portion.

86. The needle injection catheter, as described in claim 65, wherein said needle portions comprise openings along their exterior length, said openings extending from said proximal electrode to said distal electrode.

87. The needle injection catheter, as described in claim 85, wherein each of said needle portions have an additional opening at said end of said needle portion.

88. The needle injection catheter, as described in claim 86, wherein each of said needle portions have an additional opening at said end of said needle portion.
